(19) United States
(12) Patent Application Publication
(10) Pub. No.: US 2025/0050029 A1
(43) Pub. Date: Feb. 13, 2025

(54) VETERINARY SYRINGE

(71) Applicant: Henke-Sass, Wolf GmbH, Tuttlingen (DE)

(72) Inventor: Melanie EISELE, Wurmlingen (DE)

(21) Appl. No.: 18/719,730

(22) PCT Filed: Dec. 7, 2022
(86) PCT No.: PCT/EP2022/084760

§ 371 Date: Jun. 13, 2024

(30) Foreign Application Priority Data
Dec. 23, 2021 (DE) 10 2021 134 598.7

(51) Int. Cl. A61M 5/315

(52) U.S. Cl. CPC A61M 5/31511

(57) ABSTRACT

A veterinary syringe includes a syringe cylinder having a front dispensing end and a rear end. A plunger rod is arranged displaceably in the syringe cylinder. There is a fluid connection from the connection through a feed channel in the plunger rod as far as the syringe cylinder. The plunger rod has, in the front end portion thereof, a nonreturn valve which blocks the fluid connection and opens only when the pressure in the feed channel lies above the pressure in the syringe cylinder by a predetermined value. The plunger is connected releasably to the front end portion and the nonreturn valve is accessible for maintenance purposes after the plunger is detached from the end portion. An actuatable safety mechanism prevents an unintentional detaching of the plunger by detaching of the plunger from the front end portion being possible only after the safety mechanism is actuated.

### PRIORITY

This application claims the priority of German patent application DE 10 2021 134 598.7 filed Dec. 23, 2021, which is hereby incorporated herein by reference in its entirety.

### FIELD

The present invention relates to a veterinary syringe for administering, for example, medicaments.

### BACKGROUND

Frequent difficulties of known veterinary syringes are that inner parts such as, for example, nonreturn valves, have to be maintained or cleaned, but this is, however, difficult and complicated.

### SUMMARY

An object of the invention is to provide an improved veterinary syringe.

The veterinary syringe can have a main body, which comprises a syringe cylinder, having a front dispensing end, for receiving fluid to be dispensed, a plunger rod, which has a front end portion with a plunger, which is arranged displaceably along a displacement direction in the syringe cylinder, and a connection for connecting to a fluid reservoir, wherein there is a fluid connection from the connection through a feed channel in the plunger rod as far as the syringe cylinder. The plunger rod can have a nonreturn valve (preferably in the front end portion thereof), said nonreturn valve blocking the fluid connection and opening only when the pressure in the feed channel lies above the pressure in the syringe cylinder by a predetermined value. The plunger can be connected releasably to the front end portion and the nonreturn valve can be accessible for maintenance purposes after the plunger is detached from the end portion. Furthermore, an actuable safety mechanism can be formed, which prevents an unintentional detaching of the plunger by detaching of the plunger from the front end portion being possible only after the safety mechanism is actuated.

It can thereby be ensured that the plunger cannot be undesirably detached from the front end portion of the plunger rod since detaching is possible only when the safety mechanism is actuated. Maintenance of the nonreturn valve, for example, can thus be carried out, without fear of undesirable detaching of the plunger, for example when cleaning the syringe cylinder.

The detachable connection can be, for example, a screw connection, a bayonet connection or another connection.

The safety mechanism can be configured as a rotation lock which blocks rotation (e.g. unscrewing) of the plunger relative to the plunger rod (preferably about the longitudinal axis of the plunger rod).

The safety mechanism can have a securing recess as first securing element and a securing lug as second securing element, wherein, for securing purposes, the securing lug is admitted into the securing recess (the securing lug is in engagement with the securing recess) and thereby prevents the plunger from being detached from the front end portion. One of the two securing elements can be formed on the plunger and the other of the two securing elements can be formed on the front end portion.

The securing recess can be configured, for example, as a passage opening in a corresponding wall or as a type of blind hole (not extending through the entire thickness of the wall).

The plunger can have a plunger portion with a plunger surface and a hollow-cylindrical fastening portion, wherein the first securing element is formed on the wall of the hollow-cylindrical fastening portion. The first securing element can be formed, for example, at the end of the fastening portion that faces away from the plunger portion. Furthermore, the hollow-cylindrical fastening portion can have an internal thread, and the front end portion can have an external thread interacting with the internal thread.

The internal thread can have at least one thread-free region which extends (preferably rectilinearly) in the longitudinal direction of the hollow-cylindrical fastening portion. The internal thread may therefore also be referred to as an interrupted internal thread. The at least one thread-free region preferably extends in the end region of the internal thread at the end of the internal thread that faces away from the plunger portion. The thread-free region can also extend over the entire length of the internal thread.

The internal thread can have two of the thread-free regions which then preferably lie opposite each other.

The at least one thread-free region of the internal thread is preferably arranged offset in the circumferential direction relative to the first securing element.

The hollow-cylindrical fastening portion is preferably configured in such a manner that it is elastic and deformable.

The external thread can have at least one thread-free region which extends (preferably rectilinearly) in the longitudinal direction of the plunger rod. The external thread may therefore also be referred to as an interrupted external thread. The at least one thread-free region preferably extends in the end region of the external thread at the end of the external thread that faces the plunger portion. The thread-free region can also extend over the entire length of the external thread.

The external thread can have two of the thread-free regions which then preferably lie opposite each other.

In particular, the at least one thread-free region of the external thread can be arranged offset in the circumferential direction relative to the second securing element.

The plunger rod can have a rear end portion, which is positioned outside the syringe cylinder. The syringe can also have a handle, which has a front gripping part on the main body and a rear gripping part, which is fastened pivotably to the front gripping part, and a metering device for setting the fluid volume to be dispensed. The rear end portion of the plunger rod can be fastened pivotably to the rear gripping part (preferably to a portion of the rear gripping part facing the front gripping part) such that, by pivoting of the rear gripping part in the direction of the main body, the plunger rod, and therefore the plunger, is moved in the syringe cylinder to the front dispensing end in order to dispense fluid present in the syringe cylinder. The metering device can be arranged on the main body and can provide an adjustable front stop for the plunger rod, said stop defining a minimum distance between the plunger (for example the plunger surface of the plunger) and the front dispensing end that can be achieved when the front end portion of the plunger rod is moved in the syringe cylinder to the front dispensing end. This defines how far in the direction of the front dispensing end, the plunger is movable to the maximum. The adjustable front stop of the metering device can be positionable in at least two predetermined setting positions, in which the position of the front stop in the displacement direction differs, and therefore, by means of the setting of one of the setting positions, the maximum stroke of the plunger rod, and therefore the maximum fluid volume which can be dispensed, can be set.

The rear end portion of the plunger rod can be fastened pivotably to the rear gripping part such that, by pivoting of the rear gripping part in the direction of the main body, the plunger rod, and therefore the plunger, is moved in the syringe cylinder to the front dispensing end in order to dispense fluid present in the syringe cylinder. The metering device can be arranged on the main body and can provide an adjustable front stop for the plunger rod, said stop defining a minimum distance between the plunger (for example the plunger surface of the plunger) and the front dispensing end that can be achieved when the front end portion of the plunger rod is moved in the syringe cylinder to the front dispensing end. This defines how far in the direction of the front dispensing end, the plunger is movable to the maximum. The adjustable front stop of the metering device can be positionable in at least two predetermined setting positions, in which the position of the front stop in the displacement direction differs, and therefore, by means of the setting of one of the setting positions, the maximum stroke of the plunger rod, and therefore the maximum fluid volume which can be dispensed, can be set.

Since the rear end portion of the plunger rod is fastened pivotably to the rear gripping part, the plunger rod does not protrude over the rear gripping part, and therefore the rear gripping part can be configured ergonomically. Therefore, no part of the plunger rod (e.g. the rear end portion of the plunger rod) protrudes over the side of the rear gripping part facing away from the main body. In particular, the side of the rear gripping part that faces away from the main body can be configured as a continuous surface. Since the rear end portion of the plunger rod is fastenable pivotably to the rear gripping part (preferably to a portion of the rear gripping part that faces the front gripping part), the side of the rear gripping part that faces away from the main body can be configured as a continuous surface, in particular even in the portion which lies opposite the pivotable fastening of the rear end portion. Furthermore, the side of the rear gripping part that faces away from the main body can be shaped ergonomically. Of course, the front gripping part can also be configured ergonomically.

The rear gripping part can preferably be pivoted about a first pivot axis relative to the front gripping part. The first pivot axis can extend perpendicular to the displacement direction. The rear end portion of the piston rod is in particular fixed to the rear gripping part in a connecting region such that only a relative pivoting about a second pivot axis, which runs through the connecting region, is possible. This connecting region is preferably formed on a portion of the rear gripping part that faces the front gripping part. The second pivot axis can extend perpendicular to the displacement direction and/or parallel to the first pivot axis.

Owing to the adjustability of the front stop in the displacement direction, only the fluid quantity or the fluid volume which is set is ever discharged. Upon subsequent filling of the syringe cylinder, a completely filled syringe cylinder is present again and an adjustment of the front stop does not lead to any loss whatsoever of the fluid located in the syringe cylinder. The fluid can be in particular a liquid. The fluid can be a medicament, a vaccine or similar.

The metering device can have a rotary element which is mounted rotatably on the main body and comprises a stop element (e.g. a rib) for each of the predetermined injection positions. The stop elements can be arranged spaced apart from one another in the direction of rotation of the rotary element, wherein, by rotation of the rotary element, the desired stop element can be positioned as the front stop.

The maximum fluid volume which can be dispensed can therefore be reliably set in a simple manner (merely by rotating the rotary element).

The stop elements can be arranged on a helical main path which surrounds a channel through which the piston rod extends.

A compact configuration of the metering device is therefore possible.

The rotary element can have a cylindrical latching portion with grooves, wherein each groove is assigned to one of the stop elements, and the main body has an engagement element which, in the predetermined setting positions, is in engagement with one of the grooves. The desired stop position can therefore be reliably selected and set.

The grooves of the latching portion can be formed on an outer circumferential surface of the rotary element (e.g. a cylinder portion).

The plunger rod can have a plunger rod stop which, when the minimum distance is reached, is in contact with the front stop. The plunger rod stop can be web-shaped and can extend in the longitudinal direction of the plunger rod and radially.

The plunger rod stop can have a stop surface which is, for example, curved concavely. In this case, it is preferred that the stop surfaces provided by the stop elements are curved convexly such that reliable contact of the two stop surfaces of the plunger rod stop, on the one hand, and of the correspondingly selected stop element, on the other hand, is thereby ensured. Of course, it is also possible for the stop surfaces of the stop elements to be configured concavely and the stop surface of the plunger rod stop to be configured convexly. Furthermore, it is possible for the stop surfaces to each be flat.

The rear end portion of the plunger rod is in particular fastened pivotably to the rear gripping part in such a manner that a relative pivoting movement between the plunger rod and the rear gripping part is the only possible relative movement between the plunger rod and the rear gripping part. Therefore, for example, a relative movement in and transversely with respect to the longitudinal direction of the plunger rod between the plunger rod and the rear gripping part is not possible. Such a pivotable fastening of the rear end portion of the plunger rod to the rear gripping part may also be referred to as a fixed plunger rod mounting.

The plunger rod can be guided in the main body (in particular the plunger in the syringe cylinder) in such a manner that the plunger rod (or the plunger) is displaceable or movable only along the displacement direction. By means of the fixed plunger rod mounting, a movement component transversely with respect to the displacement direction (or transversely with respect to the longitudinal direction of the plunger rod) can therefore occur at the rear end portion of the plunger rod when the rear gripping part is pivoted towards the front gripping part. This results from the fact that, when the rear gripping part is pivoted, the connecting region of the pivotable fastening to the rear gripping part is moved on a circular arc, with the centre point of the circular arc lying on the pivot axis of the two gripping parts. The plunger rod can therefore be provided in particular with such elasticity (or, e.g., flexibility) that said movement component transversely with respect to the displacement direction is compensated for. This compensation can therefore be achieved in particular by the movement component transversely with respect to the displacement direction leading to bending or deflection of the plunger rod, which is possible because of the elasticity provided for the plunger rod.

The handle can be configured in such a manner that a spring element which pushes the two gripping parts apart is provided. A basic position of the veterinary syringe can therefore be provided and, for use of the syringe, a user has to move the rear gripping part in the direction of the front gripping part counter to the spring force.

In particular, the syringe (in particular the main body or a main portion of the main body) can have a rear stop which limits a movement of the plunger rod from the front dispensing end counter to the displacement direction. The position of the rear stop is preferably fixed and cannot be changed, and therefore the position of the rear stop defines the basic position of the syringe (or the maximum possible pivoting of the rear gripping part with respect to the front gripping part).

The syringe according to certain embodiments can have a connector for a fluid reservoir, wherein the connector is fluidically connected to the syringe cylinder via a fluid connection. A nonreturn valve can be arranged in the fluid connection to the syringe cylinder, said nonreturn valve blocking the fluid connection and opening only when the pressure in the fluid connection upstream of the nonreturn valve lies above the pressure in the syringe cylinder by a predetermined value.

The connector for connecting a fluid reservoir can be formed on the rear gripping part. There can be a fluid connection to the syringe cylinder from the connection through a feed channel extending through the plunger rod.

Furthermore, the front dispensing end can be fluidically connected to a front end of the syringe via a dispensing channel and a nonreturn valve can be arranged in the dispensing channel, said nonreturn valve opening only when the pressure in the cylinder exceeds a predetermined value (relative to the external pressure).

The veterinary syringe is therefore preferably configured as a self-filling syringe since, after the predetermined fluid volume is dispensed via the nonreturn valve in the dispensing channel, the rear gripping part is moved away again from the dispensing end by the pretensioning of the spring. This also leads to a corresponding movement of the plunger, as a result of which a negative pressure is generated in the syringe cylinder, leading to opening of the nonreturn valve in the plunger rod such that, owing to the now free fluid connection from the syringe cylinder as far as the fluid reservoir, fluid can be sucked out of the fluid reservoir into the syringe cylinder. As soon as the plunger rod has moved as far as the rear stop of the syringe, the nonreturn valve in the plunger rod closes again and the syringe cylinder is completely filled again.

However, any other design for providing a self-filling syringe is also possible. Thus, a connection for a fluid reservoir can also be provided on the main body or on the syringe cylinder itself, said connection being closed via a nonreturn valve which opens only when there is negative pressure in the syringe cylinder. In this case, it is not necessary for a feed channel to be formed in the plunger rod.

It will be appreciated that the features mentioned above and the features still to be explained below can be used not only in the specified combinations but also in other combinations or on their own, without departing from the scope of the present invention.

The invention is explained in even more detail below on the basis of exemplary embodiments, with reference being made to the appended drawings, which likewise disclose features essential to the invention. These exemplary embodiments are only illustrative and should not be construed as restrictive. For example, a description of an exemplary embodiment with a multiplicity of elements or components should not be construed as meaning that all of these elements or components are necessary for implementation. Rather, other exemplary embodiments can also contain alternative elements and components, fewer elements or components, or additional elements or components. Elements or components of different exemplary embodiments can be combined with one another, unless stated otherwise. Modifications and variations that are described for one of the exemplary embodiments can also be applicable to other exemplary embodiments. In order to avoid repetitions, the same elements or corresponding elements in different figures are designated by the same reference signs and are not explained several times over.

### BRIEF DESCRIPTION OF THE DRAWINGS

FIG. 1 shows a sectional view of an embodiment of the veterinary syringe;

FIG. 2 shows an isometric view of the plunger rod together with plunger;

FIG. 3 shows a front view of plunger rod and plunger;

FIG. 4 shows a sectional view of the plunger rod together with plunger along the section line A-A in FIG. 3;

FIG. 5 shows an enlarged view of the detail C from FIG. 4;

FIG. 6 shows a sectional view of the plunger rod 8 together with plunger along the section line B-B in FIG. 4;

FIG. 7 shows an isometric view of the plunger rod;

FIG. 8 shows a front view of the plunger rod from FIG. 7;

FIG. 9 shows a sectional view of the plunger rod along the section line A-A in FIG. 8;

FIG. 10 shows an isometric view of the plunger;

FIG. 11 shows a front view of the plunger;

FIG. 12 shows a sectional view of the plunger along the section line A-A in FIG. 11;

FIG. 13 shows a top view of the connecting piece;

FIG. 14 shows an isometric view of the connecting piece;

FIG. 15 shows a front view of the connecting piece;

FIG. 16 shows a sectional view of the connecting piece along the section line A-A in FIG. 15;

FIG. 17 shows a front view of the rear gripping part;

FIG. 18 shows a sectional view of the rear gripping part along the section line A-A in FIG. 17;

FIG. 19 shows an isometric view of the rear gripping part;

FIG. 20 shows an isometric view of the rotary element;

FIG. 21 shows a top view of the rotary element, and

FIG. 22 shows a sectional view of the rotary element from FIGS. 20 and 21.

### DETAILED DESCRIPTION

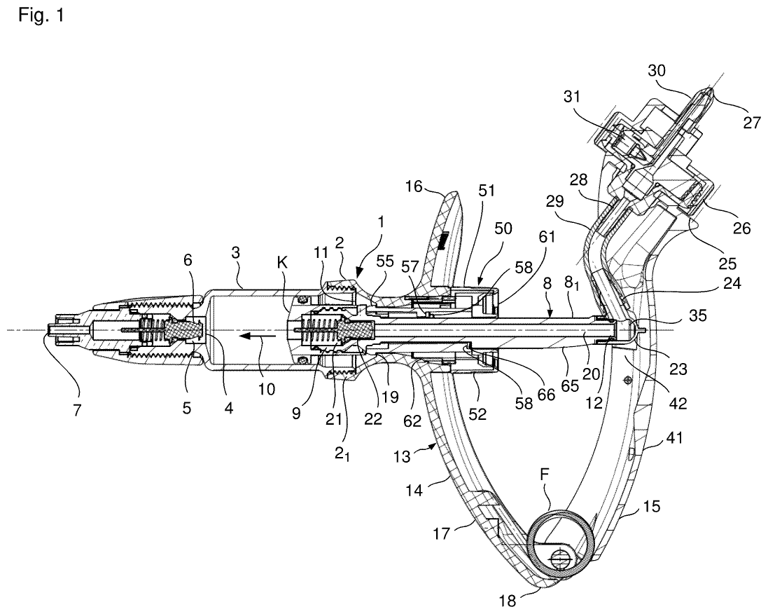

In the embodiment shown in FIG. 1, the veterinary syringe 1 comprises a main body 2 with a syringe cylinder 3 for receiving fluid to be dispensed (e.g. a medicament or vaccine in liquid form). The syringe cylinder 3 is connected detachably here to a main portion 2₁ of the main body 2 by means of a screw connection and comprises a front dispensing end 4 which opens into a dispensing channel 5 in which a first nonreturn valve 6 sits. It is possible for, for example, a cannula, not shown, to be attached to the front end 7 of the dispensing channel 5.

The syringe 1 furthermore comprises a plunger rod 8 which, with its front end portion 9, is arranged displaceably along a displacement direction 10 in the syringe cylinder 3. A plunger K is arranged at the front end portion 9, said plunger, upon displacement in the direction of the front dispensing end 4, bringing about dispensing of fluid out of the syringe cylinder 3 via the front dispensing end 4 and the dispensing channel 5 since the first nonreturn valve 6 opens because of the pressure built up in the syringe cylinder 3 by the plunger movement.

The plunger rod 8 extends out of the main body 2 beyond a rear end 11 of the syringe cylinder 3 and, as will be described in detail below, is connected by its rear end portion 12 in an articulated manner to a rear gripping part 15 of a handle 13 of the syringe 1.

The handle 13 has a front gripping part 14 formed on the main body 2 (here in the main portion 21) and the rear gripping part 15. The front gripping part 14 has an upper portion 16 protruding upwards from the main body 2 and a lower portion 17 protruding downwards from the main body 2. At the free end 18 of the lower portion 17 of the front gripping part 14, the rear gripping part 15 is mounted pivotably about a first pivot axis (which here runs perpendicular to the plane of the drawing of FIG. 1), wherein a spring F provided between the two gripping parts 14 and 15 pushes the two gripping parts 14 and 15 apart. As can be readily seen in the sectional illustration of FIG. 1, the handle 13 is substantially in the shape of a V.

In the basic position of the syringe 1 that is shown in FIG. 1, the front end portion 9 of the plunger rod 8 lies against a rear stop 19 formed in the main portion 2₁ and therefore cannot be moved further away from the front dispensing end 4 counter to the displacement direction 10. Owing to the spring F, this basic position is present if an external force is not exerted on the rear gripping part 15.

As can be gathered in particular from FIGS. 1, 4, 5, 6 and 9, the plunger rod 8 comprises a plunger rod main portion 8₁ with a feed channel 20 extending through the plunger rod main portion 8₁ in the longitudinal direction of the plunger rod 8 and being fluidically connected to the interior of the syringe cylinder 3 via a connecting channel 21 in the front end portion 9 and a passage opening 70 in the plunger K, with a second nonreturn valve 22 being arranged in the connecting channel 21.

In order to be able to fit the second nonreturn valve 22 in the front end portion 9 and to be able to, for example, clean it for maintenance purposes, the plunger K is fastened detachably to the front end portion 9 via a screw connection.

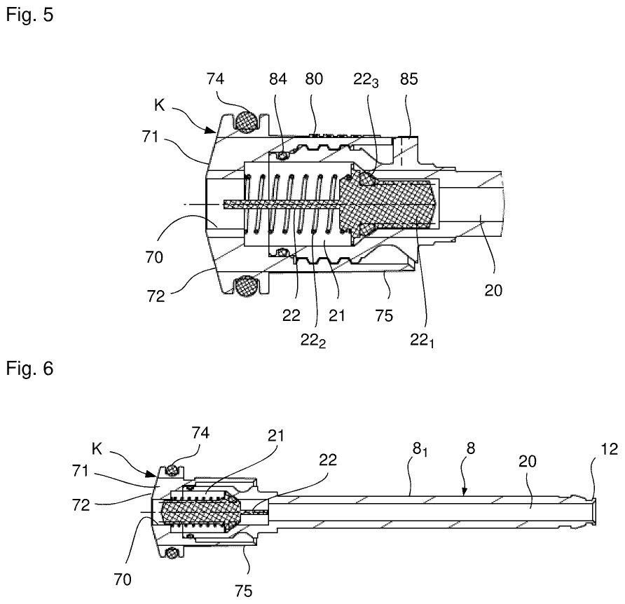

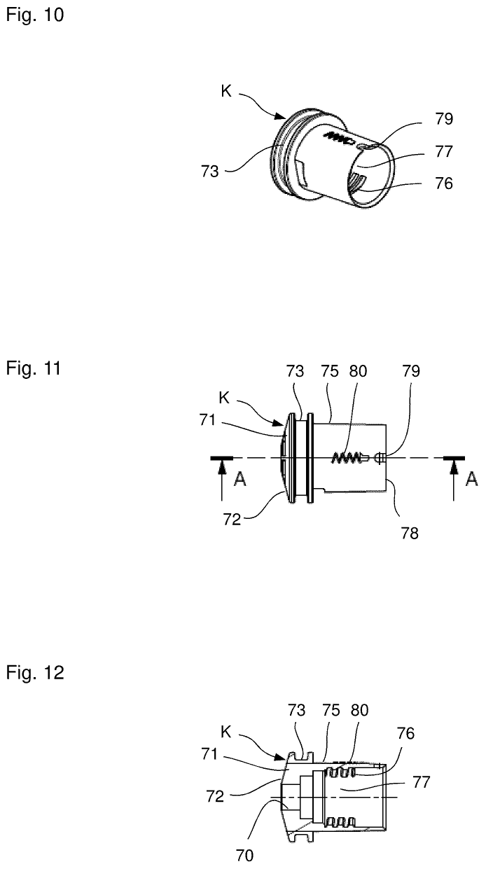

In detail, the plunger K, as can be gathered in particular from FIG. 5, comprises a plunger portion 71 with a plunger surface 72 and an annular plunger groove 73 in which a plunger sealing ring 74 sits. The plunger portion 71 is adjoined by a hollow-cylindrical fastening portion 75 which has an internal thread 76 which is not configured continuously in the circumferential direction, but rather has two opposite thread-free regions 77 (FIGS. 10 and 12).

In the wall of the hollow-cylindrical fastening portion 75, a securing recess 79 (or first securing element) is formed at the end 78 that faces away from the plunger portion 71. Furthermore, a mark 80 is also provided on the wall of the hollow-cylindrical fastening portion 75, the mark indicating how the second nonreturn valve 22 should be inserted into the connecting channel 21.

Since the passage opening 70 extends through the entire plunger portion 71 as far as the interior of the hollow-cylindrical fastening portion 75, the desired fluid connection to the supply channel 20 to the interior of the syringe cylinder 3 can be provided when the plunger K is screwed onto the front end portion 9.

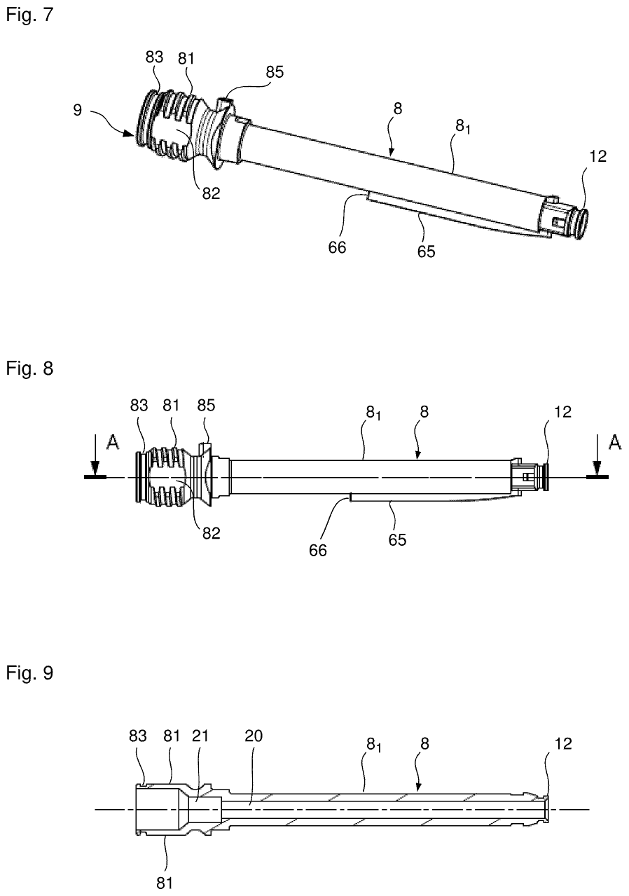

For the screwing-on operation, an external thread 81 is formed on the outer side of the front end portion 9, the external thread being interrupted in the circumferential direction by two thread-free regions 82 which preferably lie opposite each other (FIGS. 7-9). An annular groove 83 in which a sealing ring 84 sits (FIG. 5) is formed at the front end of the front end portion 9.

Furthermore, the front end portion 9 comprises a radially protruding securing lug 85 (or second securing element) which is spaced apart from the external thread 81 (FIGS. 7 and 8) in the direction of the rear end portion 12 of the plunger rod 8.

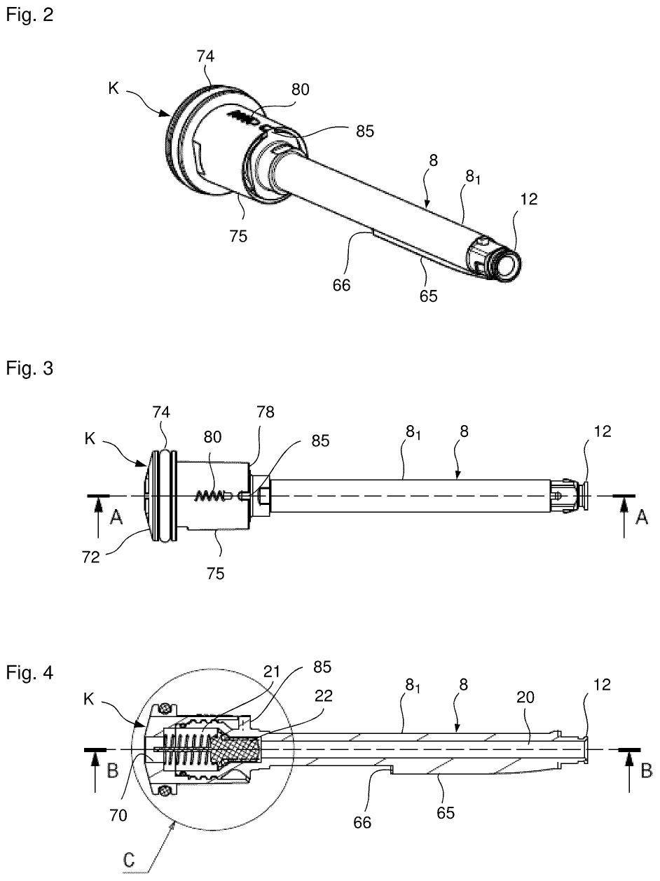

The dimensioning of the thread-free regions 82 in the circumferential direction of the external thread 81 is selected in conjunction with the dimensioning of the thread-free regions 77 of the internal thread 76 of the plunger K in such a manner that the plunger K can be screwed onto the front end portion 9 and a permanent threaded connection is possible. All that needs to be done before the end 78 of the hollow-cylindrical fastening portion 75 strikes against the securing lug 85 during the screwing-on operation is for the hollow-cylindrical fastening portion 75 to be pressed together in the regions of the thread-free regions 77. Owing to the resulting deformation of the fastening portion 75, the end 78 of the hollow-cylindrical fastening portion 75 can be moved over the securing lug 85 during the screwing-on operation, and the securing recess 79 can be positioned such that, after the described pressing together is ended, the hollow-cylindrical fastening portion 75 relaxes into its main shape because of its elasticity (the deformation of the hollow-cylindrical fastening portion 75 is thus made reversible). Since the plunger K is positioned in such a manner that the securing recess 79 lies opposite the securing lug 85, the securing lug 85 is then admitted into the securing recess 79, as shown in FIGS. 2 to 6. The securing lug 85 therefore engages in the securing recess 79, as a result of which there is protection against rotation or fixing against rotation.

Even if a rotary force is exerted on the plunger K, an undesirable unscrewing of the plunger K from the front end portion 9 can be reliably prevented since the securing lug 85 is admitted into the securing recess 79 and therefore unscrewing of the plunger K from the front end portion 9 is reliably prevented. An undesirable rotary force on the plunger K can occur, for example, when unscrewing the cylinder 3 from the main portion 2₁ of the main body 2 for cleaning purposes if, for example, due to soiling, the friction between cylinder 3 and plunger K is higher than the friction between plunger K and front end portion 9.

If, following use of the syringe 1, the second nonreturn valve 22 has to be cleaned or maintained, the cylinder 3 can first of all be unscrewed from the main portion 2₁ of the main body 2. Owing to the rotation lock by means of the securing lug 85 and the securing recess 79, the plunger K remains here on the front end portion 9. In order now to detach the plunger K from the front end portion 9, the hollow-cylindrical fastening portion 75 has to be deformed by being compressed in the region of the two opposite thread-free regions 77 such that the cross-sectional shape of the hollow-cylindrical fastening portion 75 is no longer circular but oval in the region of the securing recess 79. As a result, the securing recess 79 is moved away from the front end portion 9 along the radial direction of extent of the guide lug 85 such that there is no longer engagement of the securing lug 85 with the securing recess 79. In this state, the unscrewing of the plunger K can be begun. The dimensioning of the securing lug 85 and of the securing recess 79 in the longitudinal direction of the plunger rod 8 is selected in the embodiment described in such a manner that, after a full revolution (360°) of the plunger K, the end 78 of the wall of the hollow-cylindrical fastening portion 75 specifically no longer touches the securing lug 85. The plunger K can therefore be unscrewed further since there is no longer a rotation lock.

After the plunger K has been unscrewed, the second nonreturn valve 22 with its valve body 22₁, its valve spring 22₂ and its seal 22₃ is freely accessible and can be cleaned and maintained.

The plunger K can subsequently be screwed on again in the manner already described such that the desired rotation lock is present by the securing lug 85 engaging in the securing recess 79.

The rear end portion 12 of the piston rod 8 is connected to a connecting piece 23 (FIG. 1 and FIGS. 13-16) which is mounted rotatably in the rear gripping part 15 and has a tube attachment 24.

At the upper free end 25 (FIGS. 1, 17 to 19) of the rear gripping part 15 there is mounted a bottle adapter 26 with a piercing mandrel 27 (FIG. 1) which, for example, can pierce through a membrane of a medicament bottle (not shown) fastened to the bottle adapter 26 when the medicament bottle is fastened to the bottle adapter 26. The bottle adapter 26 furthermore has a tube attachment 28 which is fluidically connected to the piercing mandrel 27. Two ends of a tube 29 are pushed onto the two tube attachments 28 and 24 such that the piercing mandrel 27 (and therefore the contents of the corresponding medicament bottle) is fluidically connected to the feed channel 20 of the plunger rod 8 via the connecting piece 23.

The bottle adapter 26 also has a ventilation channel 30 which runs in the piercing mandrel 27 and is connected to the environment via a third nonreturn valve 31 in order to avoid the formation of an excessive negative pressure in the medicament bottle during the extraction of the liquid.

The syringe 1 is configured as what is referred to as a self-filling syringe which, after a discharge operation has taken place, automatically fills the syringe cylinder 3 with the quantity of discharged fluid.

A user of the syringe 1 can thus hold the handle 13 in their hand in such a manner that their index finger lies against the upper portion 16, their middle finger, their ring finger and their small finger lie against the lower portion 17 of the front gripping part 14, and the palm and thumb lie against the rear gripping part 15. If the user then presses their hand together, the user moves the rear gripping part 15 in the direction of the main body 2 counter to the force of the spring F, as a result of which the plunger rod 8 is moved with its front end portion 9, and therefore with the plunger K, along the displacement direction 10 in the syringe cylinder 3 to the front dispensing end 4. A positive pressure thereby builds up in the syringe cylinder 3, leading to the first nonreturn valve 6 opening and therefore fluid being able to be dispensed via the dispensing channel 5. If the user opens their hand again, the rear gripping part 15 is moved away from the main body 2 because of the spring force of the spring F, as a result of which the plunger rod 8 together with the front end portion 9 and plunger K is moved in the syringe cylinder 3 counter to the displacement direction 10. The first nonreturn valve 6 thereby closes and a negative pressure is built up in the interior of the syringe cylinder 3, leading to the second nonreturn valve 22 in the front end portion 9 opening and fluid thereby being sucked out of the medicament bottle into the interior of the syringe cylinder 3. The rearward movement of the rear gripping part 15 ends when the front end portion 9 lies against the rear stop 19. The second nonreturn valve 22 closes again and the interior of the syringe cylinder 3 is again completely filled with fluid to be dispensed.

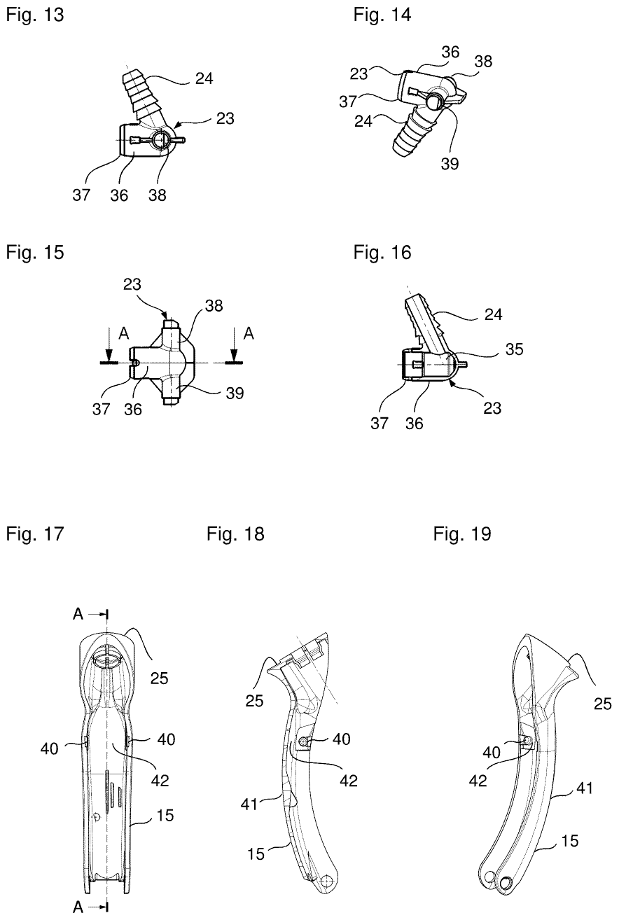

As is shown in FIGS. 13 to 16, a channel 35 formed in the connecting piece 23 is substantially V-shaped, with the connecting piece 23 having a main element 36 with a connection 37 for the rear end portion 12 of the plunger rod 8 and for the tube attachment 24 which extends obliquely with respect thereto. Two laterally extending rotary pins 38, 39 are formed on the main element 36 and are inserted into corresponding receptacles 40 (FIGS. 17-19) of the rear gripping part 15, and therefore the connecting piece 23 is mounted rotatably.

By means of this configuration of the syringe 1, the rear end portion 12 of the plunger rod 8 is therefore mounted rotatably on the rear gripping part 15 in such a manner that a longitudinal or transverse relative movement between plunger rod 8 and rear gripping part 15 is not possible. It can also be said that the rear end portion 12 of the plunger rod 8 is fixed to the rear gripping part 15 in a connecting region such that only a relative pivoting about a second pivot axis which runs through the connecting region is possible. As can be gathered in particular from FIGS. 17-19 in conjunction with FIG. 1, this connecting region is formed on a portion 42 of the rear gripping part 15 that faces the front gripping part 14.

The second pivot axis preferably extends perpendicular to the displacement direction and/or parallel to the first pivot axis about which the rear gripping part 15 can be pivoted relative to the front gripping part 14. This configuration of the fastening of the rear end portion 12 of the plunger rod 8 to the rear gripping part 15 makes it possible for the rear side 41 of the rear gripping part 15 that faces away from the main body 2 to be able to be formed ergonomically for better handling of the syringe 1. The rear side 41, and therefore also in particular the portion of the rear side 41 that is opposite the connecting region, can thus be configured continuously (without gaps or holes) and can have a corresponding ergonomic shape for a user's hand.

Since the rear end portion 12 of the plunger rod 8 is fixed to the rear gripping part 15 in the connecting region in such a manner that only a relative pivoting about the second pivot axis is possible, the plunger rod 8 is provided with a predetermined elasticity which permits the plunger rod 8 to be deflected. Thus, during pivoting of the rear gripping part 15 towards the front gripping part 14, a movement component can occur transversely with respect to the displacement direction (or transversely with respect to the longitudinal direction of the plunger rod) (e.g. from the bottom upward in FIG. 1). This results from the fact that, when the rear gripping part 15 is pivoted, the connecting region of the pivotable fastening to the rear gripping part 15 is moved on a circular arc, with the centre point of the circular arc lying on the pivot axis of the two gripping parts 14, 15. Owing to the predetermined elasticity of the plunger rod 8, the movement component transversely with respect to the displacement direction leads to bending of the plunger rod 8 such that the movement component transversely with respect to the displacement direction is compensated for. The elasticity is configured here in such a manner that the plunger rod 8 is deflected only to an extent such that the movement of the plunger rod 8 with its front end portion 9, and therefore with the plunger K, in the syringe cylinder 3 along the displacement direction 10 to the front dispensing end 4 is not affected, and therefore the plunger K is easily displaced in the syringe cylinder 3.

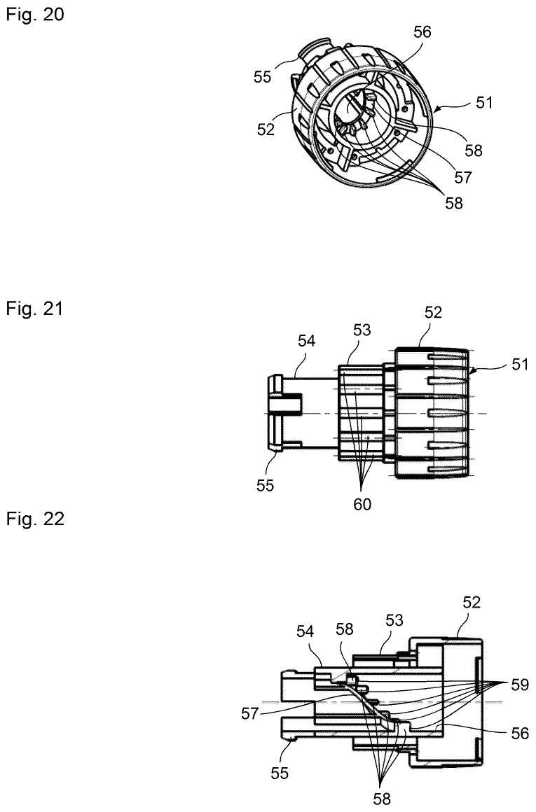

The syringe 1 furthermore has a metering device 50 which is arranged on the main body 2 and with which the maximally dispensable fluid volume can be adjusted and can be set. For this purpose, the metering device 50 comprises a rotary element 51 which is arranged at the rear end of the main body 2 and has an end cap 61 (FIG. 1). The rotary element 51 comprises a cylindrical gripping portion 52, a cylindrical latching portion 53 adjoining the latter, and a cylindrical inner portion 54 which adjoins said latching portion and has latching lugs 55, as can readily be seen in particular in FIGS. 20 to 22, with the end cap 61 not being shown in FIGS. 20 to 22. A cylindrical channel 56 through which the plunger rod 8 runs and is guided is formed in the interior of the rotary element 51. Stop elements 58 (e.g. ribs 58) which are spaced apart from one another in the circumferential direction on a helical main path 57 are formed around the channel 56 and preferably have the same height here such that their stop surfaces 59 facing away from the main path 57 are at different distances in the displacement direction 10 from the front dispensing end 4.

On the other side of the cylindrical latching portion 53, a groove 60 running in the longitudinal direction is provided for each stop element 58.

In the inserted state, which is shown in FIG. 1, the rotary element 51 is mounted rotatably on the main body 2, with the plunger rod 8 extending through the channel 56.

In the region of the cylindrical latching portion 53, the main portion 2₁ of the main body 2 has a protruding engagement element 62 which, depending on the rotation position of the rotary element 51, is in engagement with one of the longitudinal grooves 60.

As is apparent, for example, in FIGS. 1, 2, 4, 7 and 8, the plunger rod 8 has a plunger rod stop 65 which extends radially from the plunger rod main portion 8₁ and has a stop surface 66. By movement of the rear gripping part 15, the plunger rod 8 can now be moved in the direction of the front dispensing end 4 until the stop surface 66 of the plunger rod stop 65 comes into contact against the stop surface 59 of the corresponding stop element 58 on the helical main path 57. The maximum stroke of the plunger rod 8 for a discharging operation is therefore defined, as a result of which the maximum discharging volume of the fluid is also defined in the interior of the syringe cylinder 3.

If the syringe 1 is in the basic position, shown in FIG. 1, the desired maximum discharging volume can be defined by rotation of the rotary element 51 by actuation of the gripping portion 52. Said discharging volume is defined on the helical main path 57 by the stop element 58 which lies opposite the plunger rod stop 65.

The maximum discharging volume can therefore be defined in a simple manner such that it is therefore ensured that a user can always dispense the defined volume of the fluid in the syringe cylinder via the front dispensing end 4.

In the embodiment described here, for example, the stop surfaces 59 of directly adjacent stop elements can be spaced apart from one another by 1.3 mm in the displacement direction 10, as a result of which the discharging volume of 0.5 ml to 10 ml can be set in 0.5 ml steps.

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products. Moreover, features or aspects of various example embodiments may be mixed and matched (even if such combination is not explicitly described herein) without departing from the scope of the invention.

1-10. (canceled)

11. Veterinary syringe, comprising:
a main body, comprising a syringe cylinder, having a front dispensing end and a rear end, for receiving fluid to be dispensed;
a plunger rod, including a front end portion with a plunger, which is arranged displaceably along a displacement direction in the syringe cylinder; and
a connection for connecting to a fluid reservoir,
wherein there is a fluid connection from the connection through a feed channel in the plunger rod as far as the syringe cylinder,
wherein the plunger rod has a nonreturn valve which blocks the fluid connection and opens only when the pressure in the feed channel lies above the pressure in the syringe cylinder by a predetermined value,
wherein the plunger is connected releasably to the front end portion and the nonreturn valve is accessible for maintenance purposes after the plunger is detached from the end portion,
wherein an actuable safety mechanism is formed, which prevents an unintentional detaching of the plunger by detaching of the plunger from the front end portion being possible only after the safety mechanism is actuated.

12. The veterinary syringe according to claim 11,
wherein the safety mechanism has a securing recess as first securing element and a securing lug as second securing element,
wherein, for securing purposes, the securing lug is admitted into the securing recess and thereby prevents the plunger from being detached from the front end portion, and
wherein one of the two securing elements is formed on the plunger and the other of the two securing elements is formed on the front end portion.

13. The veterinary syringe according to claim 12,
wherein the plunger has a plunger portion with a plunger surface and a hollow-cylindrical fastening portion, and
wherein the first securing element is formed on the wall of the hollow-cylindrical fastening portion.

14. The veterinary syringe according to claim 13,
wherein the hollow-cylindrical fastening portion has an internal thread, and
wherein the front end portion has an external thread interacting with the internal thread.

15. The veterinary syringe according to claim 14, wherein the internal thread has at least one thread-free region which extends in the longitudinal direction of the hollow-cylindrical fastening portion.

16. The veterinary syringe according to claim 15, wherein the internal thread has two of the thread-free regions which lie opposite each other.

17. The veterinary syringe according to claim 16, wherein the at least one thread-free region is arranged offset in the circumferential direction relative to the first securing element.

18. The veterinary syringe according to claim 15, wherein the at least one thread-free region is arranged offset in the circumferential direction relative to the first securing element.

19. The veterinary syringe according to claim 14, wherein the external thread has at least one thread-free region which extends in the longitudinal direction of the plunger rod.

20. The veterinary syringe according to claim 19, wherein the at least one thread-free region is arranged offset in the circumferential direction relative to the second securing element.

21. The veterinary syringe according to claim 11, wherein the syringe is configured as a self-filling syringe.
